(19) United States
(12) Patent Application Publication
(10) Pub. No.: US 2026/0071342 A1
(43) Pub. Date: Mar. 12, 2026

(54) SYSTEM AND METHOD FOR PRODUCTION OF A FUEL FROM A CO2-RICH FLUE GAS

(71) Applicant: ALICECO IP OY, Pietarsaari (FI)

(72) Inventor: Stefan STORHOLM, Pietarsaari (FI)

(21) Appl. No.: 19/106,140

(22) PCT Filed: Aug. 25, 2023
(86) PCT No.: PCT/FI2023/050487

§ 371 Date: Feb. 24, 2025

(30) Foreign Application Priority Data
Aug. 25, 2022 (FI) 20225750

(51) Int. Cl. C25B 15/08; B01D 53/26; B01F 23/10; B01F 35/221; B01F 101/00; C07C 29/152; C10L 1/02; C25B 1/04

(52) U.S. Cl. CPC C25B 15/081; B01D 53/263; B01F 23/19; B01F 35/2217; C07C 29/152; C10L 1/02; C25B 1/04; B01D 2256/22; B01D 2257/104; B01D 2257/302; B01D 2257/404; B01D 2257/80; B01F 2101/501

(57) ABSTRACT

There is provided a system comprising burning facility (101); a synthetic fuel production facility (102); a hydrogen production facility; and an oxygen production facility (114); wherein the oxygen production facility (114) is configured to feed the produced oxygen to the burning facility (101) for combustion of fuel at the burning facility (101) using the produced oxygen, and the burning facility (101) is configured to produce a CO₂-rich flue gas based on the combustion of the fuel at the burning facility (101) using the produced oxygen, and the burning facility (101) is configured to feed the produced CO₂-rich flue gas to the synthetic fuel production facility (102) for capturing the CO₂ generated at the combustion in a fuel synthesis.

### TECHNICAL FIELD

The present invention relates to systems and methods for production of a CO₂-rich gas. More precisely, the invention relates to oxygen combustion in a burning facility using oxygen obtained as a by-product of hydrogen production.

### BACKGROUND

Carbon-neutral energy sources are of utmost importance for the industry, transport, shipping as well as for individual consumer. Hydrogen economy is one of the emerging climate-neutral ways to produce power for the ever increasing needs. Hydrogen itself, as a very easily flammable and reactive gas, is difficult to store and to transport. Therefore, other energy storage systems based on hydrogen chemistry are developed. One option is to convert the hydrogen into synthetic fuels. Synthetic fuels may be used as fuels for shipping and transport purposes.

Synthetic fuels may be synthesized from hydrogen for example in a direct carbon dioxide hydrogenation process. This process may be combined with existing carbon capture systems in e.g. power plants and industrial plants. However, the existing solutions for carbon capture are energy consuming, decreasing the net output power of e.g. a power plant, and not economically efficient. Therefore, new solutions for combining carbon capture with fuel synthesis are needed.

### SUMMARY

An object of the present invention is to provide a carbon-neutral synthetic fuel for shipping and transport purposes that is easy and safe to transport, to handle and to store.

Another object of the present invention is to reduce or even eliminate NOₓ-emissions in burning facilities.

A further object of the present invention is to provide a simple and cost-efficient system for carbon capture in burning facilities.

These objects are attained with the invention having the characteristics presented below in the independent claims. Some preferable embodiments are disclosed in the dependent claims.

The features recited in the dependent claims and the embodiments in the description are mutually freely combinable unless otherwise explicitly stated.

The exemplary embodiments presented in this text and their advantages relate by applicable parts to all aspects of the invention, both the system and the method, even though this is not always separately mentioned.

A typical system according to the invention comprises a burning facility, a synthetic fuel production facility, a hydrogen production facility, and an oxygen production facility. The oxygen production facility is configured to feed the produced oxygen to the burning facility. The produced oxygen is used for combustion of fuel at the burning facility. The burning facility is configured to produce a CO₂-rich flue gas based on the combustion of the fuel at the burning facility using the produced oxygen. The burning facility is further configured to feed the produced CO₂-rich flue gas to the synthetic fuel production facility for capturing the CO₂ generated at the combustion in a fuel synthesis.

A typical method according to the present invention comprises the following steps:
producing oxygen at the oxygen production facility;
feeding, by the oxygen production facility, the produced oxygen to the burning facility for combustion of fuel at the burning facility using the produced oxygen;
producing, at the burning facility, a CO₂-rich flue gas based on the combustion of the fuel at the burning facility using the produced oxygen;
feeding, by the burning facility, the produced dry CO₂-rich flue gas to the synthetic fuel production facility; and
capturing, at the synthetic fuel production facility, the CO₂ generated at the combustion into a synthetic fuel.

An advantage of the present invention is that capturing carbon from the CO₂-rich flue gas is significantly simplified compared to conventional combustion and carbon capture systems. With no nitrogen (N₂) or its oxides (NOₓ) in the flue gas, the need for external amine scrubbing equipment, often being complicated and energy-consuming devices, is eliminated.

Another advantage of the present invention is that synthetic fuels may be produced in a simple and cost-efficient process.

A further advantage of the present invention is that NOₓ emissions resulting from conventional combustion using air may be eliminated.

A yet further advantage of the present invention is that oxygen combustion improves energy efficiency of the combustion process over conventional air combustion. Temperature of nitrogen increases in the burning facility, resulting in a heat loss. Using oxygen instead of air as an oxidant, the heat loss associated with flow-through nitrogen in the burning facility is eliminated, resulting in an improved efficiency.

In the present examples, combustion of fuel at the burning facility is performed using combustion gas formed based on oxygen that is diluted with recirculated flue gas. The combustion process produces CO₂, whereby total amount of CO₂ in the combustion gas is lower than that in output gas of the burning facility, i.e., wet flue gas. Therefore, the total amount of CO₂ in the output gas of the burning facility is higher than the total amount of CO₂ in combustion gas. Moreover, the output gas of the burning facility may have also a high CO₂ content with respect to burning facilities, where fuel is combusted using air, where CO₂ content in the dry flue gas is typically in the range of 10-20 vol-%. Therefore, the output gas of the burning facility in the present examples may be referred to CO₂-rich flue gas.

### BRIEF DESCRIPTION OF THE DRAWINGS

FIG. 1 presents a system according to an embodiment of the invention; and

FIG. 2 presents a method according to the invention.

### DETAILED DESCRIPTION

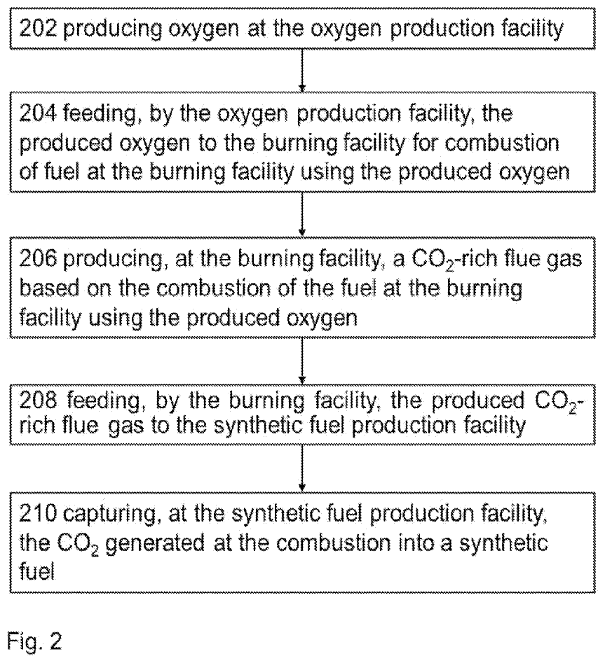

In this application, the following reference numerals will be used:
100 system
101 burning facility
102 synthetic fuel production facility
103 hydrogen production facility
104 oxygen dilution equipment
105 carbon dioxide refining equipment
106 wet scrubber
107 first control device
108 exhaust line
109 second control device
110 gas mixer
112 heat exchanger
114 oxygen production facility
202-208 steps of FIG. 2

According to the present invention, hydrogen is produced in a hydrogen production facility. The hydrogen production facility can be any facility, equipment or reaction vessel capable of producing hydrogen as a product of a chemical reaction using suitable reactants. The hydrogen may be produced e.g. by steam reforming, methane pyrolysis, partial oxidation of heavy hydrocarbons, plasma reforming, coal gasification, electrolysis, radiolysis, thermochemical methods, photocatalytic water splitting, or biocatalysed electrolysis. In a preferred embodiment, the hydrogen production facility is a water electrolysis equipment. In an embodiment, the hydrogen production facility is configured to feed the produced hydrogen to the synthetic fuel production facility 102.

According to the present invention, the system comprises an oxygen production facility configured to produce oxygen. The oxygen production facility can be any facility, equipment or reaction vessel capable of producing oxygen as a product of a chemical reaction using suitable reactants. The oxygen may be produced e.g. by air separation, such as cryogenic distillation, pressure swing adsorption, membrane separation; or oxygen evolution, such as electrolysis or chemical oxygen generation. In a preferred embodiment, the oxygen production facility is a water electrolysis equipment. The method according to the invention comprises a step of producing oxygen at the oxygen production facility.

According to the invention, the oxygen production facility is configured to feed the produced oxygen is configured to feed the produced oxygen to the burning facility 101. In an embodiment, the produced oxygen is fed to the burning facility 101 via an oxygen line. The method according to the present invention comprises a step of the feeding the produced oxygen to the burning facility 101 for combustion of fuel at the burning facility 101 using the produced oxygen.

In certain embodiments, the hydrogen production facility and oxygen production facility are located in close proximity of the burning facility 101 and the synthetic fuel production facility 102. Reactive gases, such as hydrogen and oxygen, require expensive means for transporting. Thus, the distance to transport hydrogen and oxygen is kept at a minimum by locating the facilities close to each other, preferably on the same power plant or industrial plant area. The distance between the hydrogen production facility, oxygen production facility, burning facility 101, and the synthetic fuel production facility 102 may be dictated by safety regulations. Within such safety regulations, it is beneficial to place the facilities as close to each other as possible.

In an embodiment, the oxygen production facility 114 is a part of the hydrogen production facility 103, and the oxygen production facility is configured to produce oxygen as a by-product of hydrogen production, and the oxygen production facility is configured to feed the produced oxygen to the burning facility 101. In an example, the oxygen production facility is a part of the hydrogen production facility 103, when the oxygen production facility is integrated into the hydrogen production facility 103. The oxygen production facility is integrated into the hydrogen production facility 103 for example, when the produced oxygen is obtained from the same process input raw material, e.g. water, where the hydrogen is obtained from. On the other hand the oxygen production facility is integrated into the hydrogen production facility 103, when the oxygen production and the hydrogen production are performed in parallel and the production of oxygen cannot take place without the production of hydrogen. A method according to the embodiment comprises a step of producing, at the hydrogen production facility 103, oxygen as a by-product of the hydrogen production. In an example, the oxygen production facility is configured to feed the produced oxygen to the burning facility 101 via an oxygen line.

In certain embodiments, the hydrogen production facility 103 is a part of an electrolysis equipment, wherein hydrogen is produced through electrolysis of water. The produced hydrogen is fed to the synthetic fuel production facility 102 as a raw gas for fuel synthesis. The electrolysis equipment also produces oxygen as a by-product. The produced oxygen is fed to the burning facility 101 for combustion of fuel using the produced oxygen. Conventional water electrolysis techniques may be used. The electrolysis equipment may be, for example, a polymer electrolyte membrane (PEM) cell, a solid oxide electrolysis cell, or an amine electrolysis cell. The electrolysis reaction produces hydrogen gas at the cathode and oxygen gas at the anode. The electrolysis equipment may produce, e.g., 1700 kg/h hydrogen and 13600 kg/h oxygen. In an embodiment, the water electrolysis is powered by renewable electricity, preferably wind power.

According to the invention, the burning facility 101 is configured to produce a CO₂-rich flue gas based on the combustion of the fuel at the burning facility 101 using the produced oxygen. The method according to the invention comprises a step of producing, at the burning facility 101, a CO₂-rich flue gas based on the combustion of the fuel at the burning facility 101 using the produced oxygen. The present invention can be utilized in various different burning facilities. Suitable burning facilities may be power plant furnaces or boilers, as well as industrial plant furnaces. In certain embodiments, the burning facility may be a heat plant boiler, a power plant boiler, a combined heat and power plant (CHP) boiler, a fluidized bed boiler, a recovery boiler, a rotary kiln, a cement kiln or a lime kiln.

In certain embodiments, the system 100 further comprises an oxygen dilution equipment 104. The oxygen dilution equipment 104 is used to feed diluted oxygen to the burning facility 101. Especially in burning facilities designed for air combustion, dilution of the oxygen is of essential importance. Too high oxygen content in the burning facility 101 may increase the temperature inside the burning facility to such an extent that may destroy the burning facility. In a fluidized bed boiler, for example, feeding pure oxygen would probably melt the bed.

In certain embodiments, the fuel is a fossil fuel, such as a crude oil distillate, coal or lignite, natural gas or shale gas. In other, preferred embodiments, the fuel is a renewable fuel, preferably a biofuel, more preferably a solid fuel or biomass fuel, such as sugar-producing crops, starch-producing crops, oil-producing crops, wood-based fuel. Suitable solid fuels or biomass fuels may originate from, e.g., grass, bagasse, sugarcane, corn, rapeseed, palm, straw, hardwood, softwood, bark, or any combination thereof. In an embodiment, the fuel is a solid wood-based biomass fuel, such as bark. In other embodiments, the fuel is a waste-based fuel, preferably solid or gaseous industrial or municipal waste, such as gas from animal waste, landfill gas, gas from coal mines, sewage gas, or combustible industrial waste gas. In other embodiments, the fuel may comprise fossil fuel, renewable fuel, waste-based fuel, or any combination thereof. An advantage of the present invention is that the burning facility may be operated on full capacity irrespective of fuel characteristics. Especially with biofuels, water content of the fuel varies depending on source and season. The design of burning facilities is typically based on a certain water content of the fuel. When using fuel with a high water content, capacity of the burning facility has to be reduced due to the limited allowed pressure drop in the exhaust line of the burning facility. With the oxygen combustion combined with circulation of flue gas as the oxygen diluent, composition of the flue gas can be optimized to compensate for this limitation, thus maintaining full capacity of the plant even with wet fuel.

The burning facility 101 may simultaneously be used to generate electric power and/or heat; and/or to host a chemical reaction. The flue gas is generated as a by-product at the burning facility 101. The advantage of oxygen combustion compared to conventional air combustion is that no nitrogen oxides are generated. According to the invention, oxygen is used for combustion of fuel at the burning facility 101. Typically, oxygen is used in a stoichiometric excess compared to the fuel to ensure a complete combustion. Characteristics of the used fuel may invoke a need for the stoichiometric excess. For example, a higher oxygen excess is needed for wood fuel with a high moisture content (“wet wood”) compared to fuel with a low moisture content. If the oxygen excess is too low, or if oxygen is present in less than stoichiometric ratio to the fuel, the combustion will be incomplete, producing harmful carbon monoxide and/or elemental carbon. An oxygen excess too high, on the other hand, may affect the combustion balance in the burning facility. In a typical combustion process, the oxygen excess may be e.g. 1-10% by volume, preferably 2-5% by volume, calculated from the total volume of the dry flue gas produced upon combustion.

In certain embodiments, system 100 further comprises a wet scrubber 106 connected to an exhaust line 108 of the burning facility 101. Typical scrubbing liquids in wet scrubbers may be selected from water, aqueous solutions of sodium hydroxide, calcium hydroxide, sodium carbonate, or any combination thereof. In an embodiment, the wet scrubber 106 is a water scrubber, and the scrubbing liquid is water. In an example, the wet scrubber 106 may be integrated into the burning facility 101, or it can be a stand-alone equipment. In an example, the wet scrubber is integrated into the burning facility for example, when the exhaust line is fixed to the wet scrubber for conducting at least a part of the flue gas through the wet scrubber. On the other hand, the wet scrubber may be a stand-alone equipment, when the wet scrubber can be detached from the exhaust line without a service break of the burning facility.

The wet scrubber 106 functions as a carbon capture equipment in the system. Thus, the need of an external carbon capture equipment is eliminated. The wet CO₂-rich flue gas received from the exhaust line 108 of the burning facility 101 may be lead through the wet scrubber 106 to obtain dry CO₂-rich flue gas. The dry CO₂-rich flue gas comprises at least 70% by volume, preferably at least 90% by volume, volume carbon dioxide (CO₂), of the total volume of the dry CO₂-rich flue gas. The dry CO₂-rich flue gas may comprise 70-100% by volume, preferably 80-99% by volume, more preferably 90-99% by volume, such as 95-98% by volume carbon dioxide (CO₂), of the total volume of the dry CO₂-rich flue gas. The dry CO₂-rich flue gas may also comprise less than 10% by volume, preferably less than 5% by volume, such as 1-10% or 2-4% by volume oxygen, of the total volume of the dry CO₂-rich flue gas, due to the oxygen excess at the combustion. The dry flue gas comprises a minor water vapour content according to the dew point at the actual gas temperature. The dry CO₂-rich flue gas may also comprise trace amounts of other elements or compounds originating from the fuel, such as nitrogen, sulphur and/or their oxides.

In certain embodiments, the system 100 further comprises a carbon dioxide refining equipment 105 configured to remove traces of nitrogen, sulphur and/or their oxides, and/or oxygen from the dry CO₂-rich flue gas. The carbon dioxide refining equipment is typically located downstream of the wet scrubber 106. When the dry CO₂-rich flue gas is treated with the refining equipment 106 to remove traces of nitrogen, sulphur and/or their oxides, and/or oxygen, essentially pure carbon dioxide is obtained. After refining, the dry CO₂-rich flue gas comprises at least 99% by volume, such as 99-100% by volume CO₂, of the total volume of the dry CO₂-rich flue gas.

In certain embodiments, the system 100 further comprises a gas mixer 110. The gas mixer 110 forms an output gas based on a mixture of the dry CO₂-rich flue gas and the wet CO₂-rich flue gas. The gas mixer 110 is connected to the wet scrubber 106 for receiving the dry CO₂-rich flue gas and to the exhaust line 108 for receiving the wet CO₂-rich flue gas. With the use of the gas mixer 110, output gas comprising CO₂, water, and possibly trace amounts of nitrogen, sulphur and/or their oxides, and oxygen, with a highly controllable water content may be generated. In certain embodiments, the gas mixer 110 may be placed downstream of the carbon dioxide refining equipment 105. In this example, trace amounts of nitrogen, sulphur and/or their oxides, and oxygen are removed from the gas stream before entering the gas mixer 110. Thus, the gas mixer 110 generates output gas consisting essentially of CO₂ and water, with a highly controllable water content.

In certain embodiments, the gas mixer 110 is connected to the oxygen dilution equipment 104. Thus, the output gas from the gas mixer 110 may be fed to the oxygen dilution equipment 110. The oxygen dilution equipment 104 then dilutes the oxygen received from the oxygen production facility 114 using the output gas generated at the gas mixer. The advantage of the connection between the gas mixer 110 and oxygen dilution equipment 104 lies in the circulation of the flue gas back to the burning facility 101 as the oxygen diluent. The output gas, originating from the flue gas of the burning facility 101, consists entirely of combustion products. Therefore, the output gas is an inert oxygen diluent that does not react at the combustion. Compared to conventional air oxidant, the oxygen diluted with the output gas does not produce any nitrogen oxides at the combustion. The oxygen content in a combustion chamber of the burning facility 101 can be precisely determined by determining the ratio of the output gas to the oxygen at the oxygen dilution equipment 104.

In certain embodiments, the system comprises a first control device 107 operatively connected to the oxygen dilution equipment 104 and the burning facility 101. The first control device 107 is configured to measure one or more operational characteristics of the burning facility 101 and/or the exhaust line 108. The one or more operational characteristics, such as pressure, temperature, flow rate of combustion gas, carbon monoxide concentration, oxygen concentration, or any combination thereof, may be measured at one or more points within burning facility 101 and/or at the exhaust line 108. Preferably, the one or more operational characteristics are measured at multiple points within the burning facility 101 and/or at the exhaust line 108 to create a combustion profile for the burning facility 101.

The first control device 107 controls dilution of oxygen received by the oxygen dilution equipment 104 based on the measured one or more operational characteristics of the burning facility 101 and/or the exhaust line 108. Controlling the oxygen dilution enables a precise regulation of the combustion process at the burning facility 101. In an optimal situation, the combustion profile of the burning facility may be maintained identical to conventional combustion with air. Thus, no technical modifications to the burning facility 101 itself are needed.

In certain embodiments, the system comprises a second control device 109 operatively connected to the gas mixer 110. The second control device controls a volume ratio of the wet flue gas to the dry flue gas at the gas mixer for forming the output gas. The volume ratio of the wet flue gas to the dry flue gas may be varied according to different needs. The volume ratio of the wet flue gas to the dry flue gas may vary from 100:0 to 0:100 [vol-%:vol-%], such as 100:0, 90:10, 80:20, 70:30, 60:40, 50:50, 40:60, 30:70, 20:80, 10:90, or 0:100 [vol-%:vol-%].

In certain embodiments, the second control device 109 is configured to control the volume ratio of the wet flue gas and the dry flue gas at the gas mixer for forming the output gas based on determining a need to control a combustion temperature of the burning facility 101. The wet scrubbing process decreases the temperature of the flue gas. Thus, in case the temperature of the burning facility 101 needs to be decreased, a larger amount of dry flue gas may be used to form the output gas, and the volume ratio of the wet flue gas to the dry flue gas may be 50:50, 40:60, 30:70, 20:80, 10:90 or 0:100 [vol-%:vol-%]. On the other hand, leading the hot, wet flue gas back to the burning facility 101 keeps the temperature decrease at the burning facility 101 to a minimum. Thus, if needed, the volume ratio of the wet flue gas to the dry flue gas may be 100:0, 90:10, 80:20, 70:30, 60:40, or 50:50 [vol-%:vol-%].

A yet another advantage of the present invention is that it provides corrosion protection in the burning facility 101. For example in burning facilities utilizing solid biomass fuel, such as wood, water in the fuel may cause severe corrosion due to partial condensing of water vapor at the cold end of the flue gas draft of the burning facility. The corrosion effect is pronounced when the fuel is very wet during rainy seasons, or contains even snow during winter. Using the present invention, the combustion process in the burning facility may be continuously controlled. In case of wet fuel, the ratio of wet flue gas to dry flue gas at the gas mixer can be directed to a majority of dry flue gas, even to 100 vol-% dry flue gas. The dry flue gas may be recirculated to the back to the combustion process to compensate for the additional water intake originating from the wet fuel.

In certain embodiments, the second control device 109 is configured to control the volume ratio of wet flue gas and dry flue gas at the gas mixer for forming the output gas based on determining a need to feed dry flue gas to the synthetic fuel production facility 102. The synthetic fuel production facility 102 may require dry CO₂ for an efficient reaction. Thus, it may be beneficial to keep the volume ratio of wet flue gas to dry flue gas towards a majority of dry flue gas, e.g., 50:50, 40:60, 30:70, 20:80, 10:90, or even 0:100 [vol-%:vol-%].

According to the invention, the CO₂ generated at the combustion is captured in a fuel synthesis at the synthetic fuel production facility 102. The synthetic fuel may be selected from low-molecular weight aliphatic hydrocarbons or alcohols, such as methane, methanol, ethane, ethanol, propane, propanol, butane, butanol; and biodiesel. Synthetic fuels may be used e.g. as a traffic fuel, for transportation or shipping purposes.

In certain embodiments, the synthetic fuel is methanol (CH₃OH), synthesized in a direct CO₂ hydrogenation process according to the following reactions:

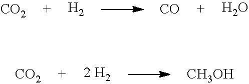

Compared to hydrogen, methanol is easier and safer to transport, to handle and to store. The need for pressurized containers is eliminated.

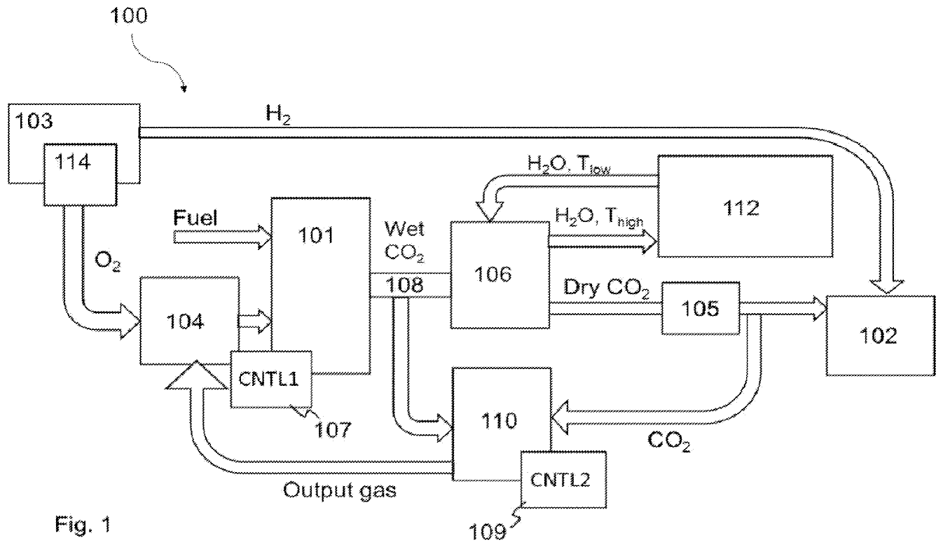

FIG. 1 presents a schematic diagram of a system 100 according to an embodiment of the present invention. The system comprises a hydrogen production facility 103, an oxygen production facility 114, a synthetic fuel production facility 102, a burning facility 101, an oxygen dilution equipment 104, a first control device 107 operatively connected to the oxygen dilution equipment 104 and the burning facility 101, an exhaust line 108, a wet scrubber 106, a carbon dioxide refining equipment 105, a gas mixer 110, a second control device 109 operatively connected to the gas mixer 110, and a heat exchanger 112. The heat exchanger 112 may be used to connect the system 100 into a heat-consuming facility, e.g. to a district heating network.

FIG. 2 presents a schematic diagram of the method according to the present invention. The method comprises producing 202, at the hydrogen production facility 103 by the oxygen production facility 114, oxygen as a by-product of the hydrogen production; feeding 204, by the hydrogen production facility 103, the produced oxygen to the burning facility 101 for combustion of fuel at the burning facility 101 using the produced oxygen; producing 206, at the burning facility 101, a CO₂-rich flue gas based on the combustion of the fuel at the burning facility 101 using the produced oxygen; feeding 208, by the burning facility 101, the produced CO₂-rich flue gas to the synthetic fuel production facility 102; and capturing 210, at the synthetic fuel production facility 102, the CO₂ generated at the combustion into a synthetic fuel.

In an example in accordance with at least some embodiments, a control device may be operatively connected to one or more equipment of a system 100, for example one or more of an oxygen dilution equipment 104, a burning facility 101, a gas mixer 110, a synthetic fuel production facility 102, an oxygen production facility 114, a carbon dioxide refining equipment, a wet scrubber 106, and other device(s) for receiving and sending information for example messages comprising measurements and/or control commands. Accordingly, the control device may send control commands to one or more of the oxygen dilution equipment 104, the burning facility 101, the gas mixer 110, the synthetic fuel production facility 102, the oxygen production facility 114, the carbon dioxide refining equipment, the wet scrubber 106, and the other device(s). On the other hand, the control device may receive information such as measurements from one or more of the oxygen dilution equipment 104, the burning facility 101, the gas mixer 110, the synthetic fuel production facility 102, the oxygen production facility 114, the carbon dioxide refining equipment, the wet scrubber 106, and the other device(s). Examples of the measurements comprise temperature measurements, pressure measurements and content of flue gas. Content of the flue gas may be measured for example regarding content of carbon monoxide, content of oxygen and/or content of CO₂, whereby burning at the burning facility may be monitored. Examples of other device(s) of the system comprise may be sensors for example one or more of temperature sensors, pressure sensors, oxygen sensors, carbon monoxide sensors and CO₂ sensors. The other device(s) may be deployed to the system for measuring operation of the oxygen dilution equipment 104, the burning facility 101, the gas mixer 110, the synthetic fuel production facility 102, the oxygen production facility 114, the carbon dioxide refining equipment and/or the wet scrubber 106. It should be noted that instead of having a single control device connected to the one or more of the oxygen dilution equipment 104, the burning facility 101, the gas mixer 110, the synthetic fuel production facility 102, the oxygen production facility 114, the carbon dioxide refining equipment and the wet scrubber 106, one or more further control devices may be provided. For example, one control device may be connected to the gas mixer 110 and optionally to other device(s) such as a sensor configured to measure operation of the gas mixer. Another control device may be connected to the oxygen dilution equipment 104 and the burning facility and optionally to other device(s) such as a sensor configured to measure operation of the oxygen dilution equipment 104 and/or the burning facility. In an example, communications between a control device and the oxygen dilution equipment 104, the burning facility 101, the gas mixer 110, the synthetic fuel production facility 102, the oxygen production facility 114, the carbon dioxide refining equipment, the wet scrubber 106 and/or the other device(s) may be digital communications for example over a wired or wireless connection. Examples of the connections comprise field bus technologies such as Profibus, Scanbus, Internet Protocol and Ethernet connections. In an example, the control device may comprise memory that stores instructions that when executed by the control device cause one or more functionalities described with an example and/or embodiment described herein.

In an embodiment an apparatus, or a control device, comprises at least one processor and a communications unit, for example a transceiver. The processor is operatively connected to the communications unit for controlling the communications unit. The apparatus may comprise a memory. The memory may be operatively connected to the processor. It should be appreciated that the memory may be a separate memory or included to the processor and/or the transceiver. The memory may store instructions that, when executed by the at least one processor causes execution of one or more functionalities in accordance with a method described herein. In an example, the transceiver is configured to perform digital communications for example over a wired or wireless connection. Examples of the connections comprise field bus technologies such as Profibus, Scanbus, Internet Protocol and Ethernet connections.

Embodiments may be implemented in software, hardware, application logic or a combination of software, hardware and application logic. The software, application logic and/or hardware may reside on memory, or any computer media. In an example embodiment, the application logic, software or an instruction set is maintained on any one of various conventional computer-readable media. In the context of this document, a “memory” or “computer-readable medium” may be any media or means that can contain, store, communicate, propagate or transport the instructions for use by or in connection with an instruction execution system, apparatus, or device, such as a computer.

Reference to, where relevant, “computer-readable storage medium”, “computer program product”, “tangibly embodied computer program” etc., or a “processor” or “processing circuitry” etc. should be understood to encompass not only computers having differing architectures such as single/multi-processor architectures and sequencers/parallel architectures, but also specialized circuits such as field programmable gate arrays FPGA, application specify circuits ASIC, signal processing devices and other devices. References to computer readable program code means, computer program, computer instructions, program instructions, instructions, computer code etc. should be understood to express software for a programmable processor firmware such as the programmable content of a hardware device as instructions for a processor or configured or configuration settings for a fixed function device, gate array, programmable logic device, etc.

1. A system comprising:
a burning facility; a synthetic fuel production facility; a hydrogen production facility; and an oxygen production facility; wherein the oxygen production facility is configured to feed the produced oxygen to the burning facility for combustion of fuel at the burning facility using the produced oxygen, and the burning facility is configured to produce a CO₂-rich flue gas based on the combustion of the fuel at the burning facility using the produced oxygen, and the burning facility is configured to feed the produced CO₂-rich flue gas to the synthetic fuel production facility for capturing the CO₂ generated at the combustion in a fuel synthesis.

2. The system of claim 1, wherein the oxygen production facility is a part of the hydrogen production facility, and the oxygen production facility is configured to produce oxygen as a by-product of hydrogen production, and the oxygen production facility is configured to feed the produced oxygen to the burning facility.

3. The system of claim 1, further comprising:
carbon dioxide refining equipment for removing oxygen, nitrogen, and sulphur oxide traces from the CO₂-rich flue gas.

4. The system of claim 1, further comprising:
a wet scrubber connected to an exhaust line of the burning facility configured to obtain dry CO₂-rich flue gas from wet CO₂-rich flue gas received from the exhaust line.

5. The system of claim 4, further comprising:
a gas mixer configured to form an output gas based on a mixture of the dry CO₂-rich flue gas and the wet CO₂-rich flue gas, whereby the gas mixer is connected to the wet scrubber for receiving the dry CO₂-rich flue gas and to the exhaust line for receiving the wet CO₂-rich flue gas.

6. The system of claim 5, further comprising:
an oxygen dilution equipment for feeding diluted oxygen to the burning facility, and the gas mixer is connected to the oxygen dilution equipment for feeding the output gas to the oxygen dilution equipment for diluting oxygen received by the oxygen dilution equipment.

7. The system of claim 6, further comprising:
a first control device operatively connected to the oxygen dilution equipment and the burning facility, wherein the first control device is configured to:
measure one or more operational characteristics of the burning facility and/or the exhaust line; and
control dilution of oxygen received by the oxygen dilution equipment based on the measured one or more operational characteristics of the burning facility and/or the exhaust line.

8. The system of claim 5, further comprising:
a second control device operatively connected to the gas mixer for controlling a volume ratio of the wet flue gas to the dry flue gas at the gas mixer for forming the output gas.

9. The system of claim 8, wherein the second control device is configured to:
control the volume ratio of the wet flue gas to the dry flue gas at the gas mixer for forming the output gas based on at least one of:
determining a need to control a combustion temperature of the burning facility, and
determining a need to feed dry flue gas to a synthetic fuel production facility.

10. The system of claim 1, wherein the burning facility is a heat plant boiler, a power plant boiler, a combined heat and power plant boiler, a recovery boiler, a rotary kiln, a cement kiln or a lime kiln.

11. The system of claim 1, wherein the fuel is a fossil fuel, renewable fuel, waste-based fuel or any combination thereof, preferably wherein the fuel originates from biofuel, more preferably a solid fuel or biomass fuel, such as sugar-producing crops, starch-producing crops, oil-producing crops, solid wood-based fuel, or any combination thereof.

12. A method for a system according to claim 1, comprising:
producing oxygen at the oxygen production facility;
feeding, by the oxygen production facility, the produced oxygen to the burning facility for combustion of fuel at the burning facility using the produced oxygen;
producing, at the burning facility, a CO₂-rich flue gas based on the combustion of the fuel at the burning facility using the produced oxygen;
feeding, by the burning facility, the produced dry CO₂-rich flue gas to the synthetic fuel production facility; and
capturing, at the synthetic fuel production facility, the CO₂ generated at the combustion into a synthetic fuel.

13. The method of claim 12, comprising:
producing, at the hydrogen production facility oxygen as a by-product of the hydrogen production;
feeding, by the hydrogen production facility, the produced oxygen to the burning facility for combustion of fuel at the burning facility using the produced oxygen

14. The method of claim 13, comprising:
refining, at the carbon dioxide refining equipment, the CO₂-rich flue gas for removing traces of nitrogen, sulphur, and/or their oxides, and/or oxygen from the CO₂-rich flue gas.
